(19) United States
(12) Patent Application Publication
(10) Pub. No.: US 2003/0082163 A1
(43) Pub. Date: May 1, 2003

(54) Fused cells, methods of forming same, and therapies utilizing same

(76) Inventor: Suyu Shu, Shaker Heights, OH (US)

Correspondence Address: Pepper Hamilton LLP, One Mellon Center, 50th Floor, 500 Grant Street, Pittsburgh, PA, 15219, US

(21) Appl. No.: 10/280,377

(22) Filed: Oct. 25, 2002

Related U.S. Application Data
(60) Provisional application No. 60/348,026, filed on Oct. 26, 2001.

(51) Int. Cl. A61K 48/00; A61K 39/00; C12N 5/08

(52) U.S. Cl. 424/93.21; 424/277.1; 435/372

(57) ABSTRACT

A method and apparatus for electrofusing a plurality of cancer cell types to dendritic cells (DCs) which results in the formation of electrofused DC-tumor cells with high efficiency and high viability. The method comprises subjecting the cells with multiple pulses of voltage in specially designed media. This is the first documentation of electrofusion of large numbers of DCs and tumor cells. Evidence demonstrates those cells contain all essential DC molecules and express tumor antigens. A single vaccination of animals with pre-existing tumors results in substantial erradication of tumors and cure of animals. The electrofused-cells can be used immediately. These fusion hybrids are to be used for the treatment of cancer directly as vaccines or indirectly by activating immune lymphocytes for adoptive immunotherapy.

### CLAIM OF PRIORITY

This application claims priority to provisional U.S. patent application No. 60/348,026 entitled Electrofusion, Hybrids Formed Thereby, and Therapies Utilizing Same, filed Oct. 26, 2001 and incorporates this provisional application herein by reference thereto in its entirety.

### STATEMENT OF GOVERNMENT INTEREST

The United States Government may have certain rights to this invention pursuant to Grant No. R01 CA84110 from the National Cancer Institute.

### FIELD OF INVENTION

The present invention generally relates to fused cells, and methods of fusing cells, as well as therapies utilizing the same. More particularly, the present invention relates to hybrid cells formed by electrofusion, and therapies utilizing the electrofused cells. Even more particularly, the present invention is directed to cell or cell-like structures that are electrofused to a dendritic cell or dendritic-like cell. In a preferred embodiment a dendritic cell-tumor cell hybrid is capable of conferring tumor resistance in vitro and in vivo and mediating tumor regression in vivo. The present invention includes procedures of cancer immunotherapy, preferably by generating chimeric hybrids from dendritic or dendritic-like cells and tumor cells.

### BACKGROUND OF THE INVENTION

The introduction of pathogens such as bacteria, parasites or viruses into a mammal elicits a response contributing to the specific elimination of the foreign organism. Foreign material is referred to as an antigen (Ag), and the specific response is called the immune response. The immune response starts with the recognition of the antigen by a lymphocyte, proceeds with the elaboration of specific cellular and humoral effectors and ends with the elimination of the antigens by the specific effectors. The specific effectors are essentially T lymphocytes and antibodies, mediating cellular and humoral immune responses, respectively. One aspect of the present invention relates to the initiation of a cellular immune response to tumors. The initiation of a cellular immune response starts with the recognition of an antigen on the surface of an antigen-presenting cell (APC).

The mode of Ag presentation shapes the nature of the immune response. To elicit a therapeutic anti-tumor immune response, dendritic cells (DCs) have been employed as a cellular adjuvant with tumor Ags in the form of tumor lysates, proteins or peptides. In all these approaches, DCs were loaded with an exogenous source of Ags.

Cellular antigen recognition is operated by a subset of lymphocytes called T-lymphocytes. T-lymphocytes include two major functional subsets. They are T-helper lymphocytes (TH), that usually express the CD4 surface marker, and cytotoxic T-lymphocytes (CTL), that usually express the CD8 surface marker. Both T-cell subsets express an antigen receptor that can recognize a given peptide antigen. The peptide needs to be associated with a major histocompatibility molecule (MHC) expressed on the surface of the APC, a phenomenon known as MHC restriction. T-cells bearing the CD4 surface market recognize peptides associated with MHC class II molecules, whereas T-cells bearing the CD8 surface marker recognize peptides associated with MHC class I molecules.

Since the T-cell antigen receptor can only recognize peptides associated with MHC molecules at the surface of an APC, cellular proteins need to be processed into such peptides and transported with MHC molecules to the cell surface. This is referred to as antigen processing. Exogenous proteins, phagocytosed by the APC, are broken down into peptides that are transported on MHC class II molecules to the cell surface, where they can be recognized by CD4⁺ T-cells. In contrast, endogenous proteins, synthesized by the APC, are also broken down into peptides, but the latter are transported on MHC class I molecules to the cell surface, where they can be recognized by CD8⁺ T-cells.

When a T-cell binds through its antigen receptor to its cognate peptide-MHC complex on an APC, the binding generates a first signal from the T-cell membrane towards its nucleus. However, this first signal is insufficient to activate the T-cell, at least as measured by the induction of cytokine synthesis and secretion. Activation only occurs if a second signal or costimulatory signal is generated by the binding of other APC surface molecules to their appropriate receptors on the T-cell surface. The capacity to present peptide Ags together with costimulatory molecules in such a way as to activate T-cells is hereafter referred to as antigen presentation. Only APCs have the capacity to present antigens to CD4⁺ (predominantly TH) and CD8⁺ (predominantly CTL) T-cells, leading to the development of immune responses.

The term dendritic cells (DCs) or dendritic-like cells (DLCs) used herein refers to not only to dendritic cells of myeloid origin, but also to cultured monocytes, and other cells present in enriched or purified dendritic cell preparations. In humans, blood or bone marrow are the usual sources of DCs, that are used either immediately or more often after culture in the presence of cytokines. Several protocols of purification and in vitro culture have been published. Unless otherwise specified herein, the term dendritic cell (“DC”) and dendritic-like cell (“DC”) are used interchangeably herein. For convenience DC will be used hereafter to refer to both DCs and DLCs unless otherwise specified.

There is increasing evidence that tumor cells do not usually function as APCs. Although some tumor cells are capable of delivering an antigen-specific signal to T cells, they may not provide the signals which are necessary for the full activation of T-cells and thereby fail to induce an efficient anti-tumor immune response. In order to compensate for this inefficient induction of an anti-tumor immune response, different approaches have been tried in experimental animals.

Dendritic cells are professional APCs capable of initiating a primary T-cell immune response. They express high levels of MHC, adhesion and costimulatory molecules as well as synthesize a variety of immunologically important cytokines such as IL-I, TNFα and IL-2. DC-based strategies thus hold promise for cancer immunotherapy and are currently under intensive investigation. In animal models, vaccines have been developed by pulsing DCs with proteins or peptides derived from tumor Ags, or transducing DCs with viral vectors encoding tumor Ags. Although Ag-loaded DCs can induce anti-tumor CTL immune responses in several model systems, these approaches are limited by their dependence on the efficiency of Ag loading to exert Ag presenting biological functions and on the availability of chemically defined antigenic proteins and peptides.

Techniques have been described in the art purportedly for inducing fusion between cells. These techniques include chemical fusion employing polyethylene glycol (PEG), and use of biological fusogens such as viruses or viral proteins. Fusion by chemical means or via biological fusogens has clear limitations, including the presence of chemical or biological contaminants inherent to the technique, resistance to fusion exhibited by some cell types, low efficiency, and toxicity.

Other strategies have been developed to induce a polyclonal immune response against a broad array of both known and undefined tumor Ags. Autologous or syngeneic DCs have been exposed to whole tumor cell lysates, loaded with peptides eluted from tumor cells or transfected with RNA from tumor cells. Several studies have suggested that such loaded DCs induce protective and therapeutic immunity against tumors in vivo. However, many details have yet to be defined because DCs exhibit extensive morphological and functional plasticity. Although theoretically attractive, little information is available with regard to levels of immunogenicities of various DC products due to variations of individual technical details. This has contributed to the poor reproducibility of many published findings and a lack of consensus on approaches for optimal procedures of DC immunotherapy.

In the early literature of tumor immunology, one of the dominant methods to induce tumor specific immunity was immunization with nonproliferating but viable irradiated or mitomycin C-treated whole tumor cells. Immunization with dead tumor cells or subcellular preparations was universally ineffective. This is an important issue which has received relatively little attention in recent years. If technically successful, it would be preferable for DC-tumor fusion hybrids to have the capacity to elicit both MHC class I- and II-restricted responses by endogeneously processing and presenting both known and yet unidentified tumor Ags in their unaltered forms. In most reported studies, fusion was accomplished with the use of (PEG) and unequivocal evidence of successful production of fusion hybrids has not been documented. Because fusion requires mixing of viable DCs and tumor cells in the same cell suspension, this co-mingling of the two cells may result in heightened immunogenicity of the tumor due to Ag uptake and presentation by DCs or the presence of enhanced costimulation when inoculated into animals. In fact, the immunogenicity of tumor cells could be improved by mere co-administration or intratumoral injection of DCs. Therefore, studies of the immunogenic potential of DC-tumor fusion hybrids in the absence of stringent and unequivocal documentation of hybrid cell production could lead to an erroneous interpretation of experimental results.

Somatic cell fusion is an old concept and practice which has played an important role in diverse areas of biological research including genetics, developmental biology and immunology. Although fusion with PEG has been the predominant method for generating mAb-producing hybridomas, the intrinsic toxicity and poor reproducibility make it difficult to be adapted for clinical cancer immunotherapy. Fusion by exposing cells to electric fields represents an attractive technique. Although traditional techniques have been sufficient for the purpose of modifying plant cells and in turn, of improving crops as well as for generation of mAb-producing hybridomas, the established procedures are not suitable for generating large numbers of hybrid cells for clinical immunotherapy.

The various embodiments of the present invention has a number of separate and distinct advantages over the related art, including: the elimination of toxic or detrimental chemical agents PEG; substantial elimination of false positive cells; high rate of fusion; production of large numbers of fused cells; obtaining large number of cells without the need to grow cells; the ability to inactivate the tumor cell prior to introduction in vivo; and the use of the fused cell without additional manipulation (e.g. purification and/or cloning).

### SUMMARY OF THE INVENTION

The present invention provides DC-tumor cell, as well as DC-tumor cell for use in the treatment of cancers. The hybrids of the invention are preferably electrofused capable of inducing an anti-tumor response when administered to a subject, in vivo. This alternative method is to engineer a hybrid cell with characteristics of both cells, for example characteristics of DCs while preserving unaltered Ags of the whole tumor cell.

In one embodiment of the invention, tumor cells are fused with DCs or DCs, and the resulting plurality of hybrids is used directly for treatment, without selection. The DC/tumor cell hybrid is administered to the subject to induce an immune response against residual tumor cells in the subject's circulation or organs. Alternatively, the hybrid is cocultivated in vitro with immune cells from the subject in order to activate against the tumor cell; the activated immune cells are then returned to the subject for cancer therapy.

This invention is more particularly in the field of immunotherapy for the treatment of cancer. Specifically, the invention provides a tumor cell electrofused to a DC. The electrofused hybrid is capable of inducing an anti-tumor response in vivo when administered to a subject in need of anti-tumor treatment (e.g. malignant gliomas renal cell carcinoma, breast and colon cancer, and melanomas).

The invention preferably provides DC-tumor cell and pluralities of DC-tumor cell hybrids that confer tumor resistance in vivo. The hybrids are generated by electrofusion of tumor cells with DCs. For instance, autologous or selected allogeneic tumor cells tumor cell lines can be electrofused with autologous or HLA-matched allogeneic DCs. Autologous tumor cell lines can be derived from primary tumors and from their metastases. Selected allogeneic tumor cell lines based on the expression of shared tumor-associated Ags can also be used as the fusion partner. DCs from an autologous or allogeneic HLA-matched individuals can be electrofused with tumor cells. DCs can be prepared from various sources such as peripheral blood and bone marrow. DC-tumor cell hybrids and pluralities of hybrids thus formed can be directly infused for active immunization of cancer patients against their residual tumors. The hybrids can also be used for the in vitro activation of autologous immune cells before their reinfusion into the patient for passive immunization against the tumor cells.

An additional embodiment of the present invention, the present invention describes the use of hybrids in combination with an immune adjuvant (e.g. IL-12, 41BB mAb or OX-40R mAb) to active immunotherapy.

### BRIEF DESCRIPTION OF FIGURES AND TABLES

FIG. 1 illustrates FACS profiles of hybrid cells generated by electrofusion. FIG. 1A illustrates the Phenotypes of DCs and D5LacZ3 tumor cells. FIG. 1B, FACS analyses of electrofusion of DCs and D5acZ3 cells. FIG. 1C, Expression of β-gal in fusion cells was detected by direct immunofluorescence staining with FDG;

FIG. 2 illustrates active immunotherapy of the D5acZ3 tumor;

FIG. 3 illustrates the survival of mice bearing 3-day established pulmonary D5acZ3 metastases following active immunotherapy with fusion cells;

FIG. 4 illustrates the specificity of active immunotherapy mediated by vaccination with DC-tumor fusion hybrid cells;

FIG. 5 illustrates the therapeutic efficacy of DC-tumor fusion. Other methods of DC loading with antigenic protein or peptide are ineffective. The experimental procedure is the same as that illustrated in FIG. 2;

FIG. 6 illustrates the activation of both CD4 and CD8 T cell responses for effective fusion cell vaccine therapy;

FIG. 7 illustrates IFN-γ secretion by activated tumor-draining lymph node (LN) T cells after stimulation in vitro with electrofusion cells;

FIG. 8 illustrates analyses of IFN-γ secretion by stimulation with DC-tumor fusion of purified CD4 or CD8 T cells from D5acZ3 tumor-draining LN cells. ELISA is the same as that described in FIG. 7;

FIG. 9 illustrates active immunotherapy of GL261 glioma with electrofused dendritic/tumor hybrids. Animals were inoculated subcutaneously with 2×10⁶ GL261 glioma cells. 3 days later they were immunized with DC-GL261 hybrids. Some animals also received IL-12 (0.2 μg i.p.) as an adjuvant for 4 days. Mice treated with fusion cells plus IL-12 demonstrated complete tumor regression;

FIG. 10 illustrates the treatment of 3-day established pulmonary metastases derived from the MCA 205 sarcoma. This experiment also illustrates the adjuvant activity of OX-40R mAb which was administered on the day of fusion vaccination at a dose of 150 μg i.p;

FIG. 11 illustrates that effective fusion cell vaccination is preferably mediated by using autologous DCs for generation of fusion hybrids. A complete mismatch of MHC for DCs did not stimulate an immune response;

FIG. 12 illustrates treatment of 3-day subcutaneously growing MCA 205 tumor by vaccination with DC-tumor electrofusion cells. Note: 6 of 10 mice treated with fusion cells plus OX-40R mAb were cured;

FIG. 13 illustrates successful treatment of 3-day established intracranial MCA 205 tumor by vaccination with DC-tumor fusion cells and OX-40R mAb;

FIG. 14 illustrates electrofusion of human DCs and Polyvalent Melanoma Cell Vaccine (PMCV) which consists of a mixture of 3 selected melanoma cell lines;

FIG. 15 illustrates electrofusion of human DCs and individual human melanoma cell lines of PMCV, M101, M10 and M24;

FIG. 16 illustrates electrofusion of another melanoma cell line, 888 Mel, with 4 distinct DC preparations;

FIG. 17 illustrates a top view of a fusion chamber in accordance with a preferred embodiment of the present invention; and

FIG. 18 illustrates a side view of the fusion chamber of FIG. 17 in accordance with a preferred embodiment of the present invention.

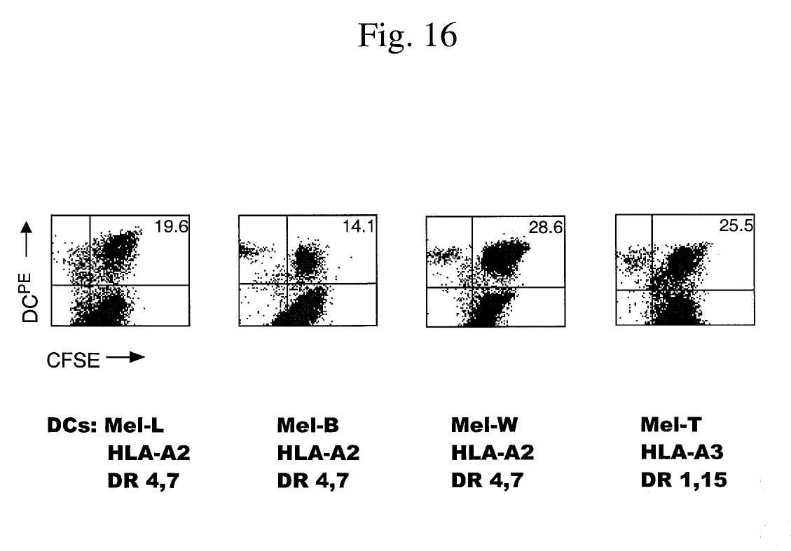

Table 1 demonstrates the ability of DC-tumor fusion hybrids generated from melanoma patient L (HLA-A2⁺, DR4⁺, and DR7⁺) and the 888 Mel melanoma cells to stimulate IFN-γ and GM-CSF secretion from four defined melanoma reactive T cells lines. Note: as illustrated in FIG. 16, fusion of 888 Mel with DCs of melanoma patient T, which was HLA-A2⁻, DR4⁻, and DR7⁻ could not stimulate these selected T cells because they are restricted by HLA-A2, DR4, or DR7.

Table 2 illustrates that only adherent fusion cells can stimulate specific IFN-γ release from a melanoma reactive T cell line, TIL 1200. Note: nonadherent fraction and mixture of DC and tumor cells could not stimulate this T cell line. Both DC's generated from melanoma patients L and W express HLA-A2, DR4, and DR7.

### DETAILED DESCRIPTION OF THE PREFERRED EMBODIMENTS

Cell hybrids have many utilities. Fusing DCs with tumor cells to develop immunogenic materials is of great value.

There are several advantages in producing cell hybrids by electrofusion. For example, fusion parameters can be easily and accurately controlled to conditions depending on the cells to be fused. Because clinical use of fused cells requires large-volume preparations and immediate use for vaccinations, a large-scale electrofusion technique with which DC-tumor hybrids were generated with high efficiency is described herein. Electrofusion of cells has shown to dramatically increase fusion efficiency over that of fusion by chemical means or via biological fusogens. Electrofusion is performed by applying electric pulses to cells in suspension. By exposing cells to an alternating electric field, cells are brought close to each other in forming pearl chains in a process termed “dielectrophoresis alignment.” Subsequent higher voltage pulses cause cells to form pores on the cell membrane termed “reversible membrane breakdown” which under normal circumstances will spontaneously reseal. However, when cells are closely adjacent to each other under dielectrophoresis, resealing will occur between adjacent cells, resulting in the fusion of different cells.

The term “exogenous macromolecules” is used herein, for purposes of the specification and claims, to mean biomolecules including, but not limited to, peptides, proteins, antigens, antibodies, cell receptors, enzymes, polysaccharides, oligonucleotides, DNA, RNA, recombinant vectors, drugs and dyes.

The term “endogenous macromolecules” is used herein, for purposes of the specification and claims, to mean biomolecules including, but not limited to, peptides, proteins, antigens, antibodies, cell receptors, enzymes, polysaccharides, naturally occurring in a cell.

The term “cells” is used herein, for purposes of the specification and claims, to mean animal cells and particularly mammalian cells.

Although the present invention has wide applicability due to its effectiveness and efficiency in fusing cells, the present invention is particularly useful in the treatment of cancer via the presentation of hybrid cancer-dendritic cells to the immune system.

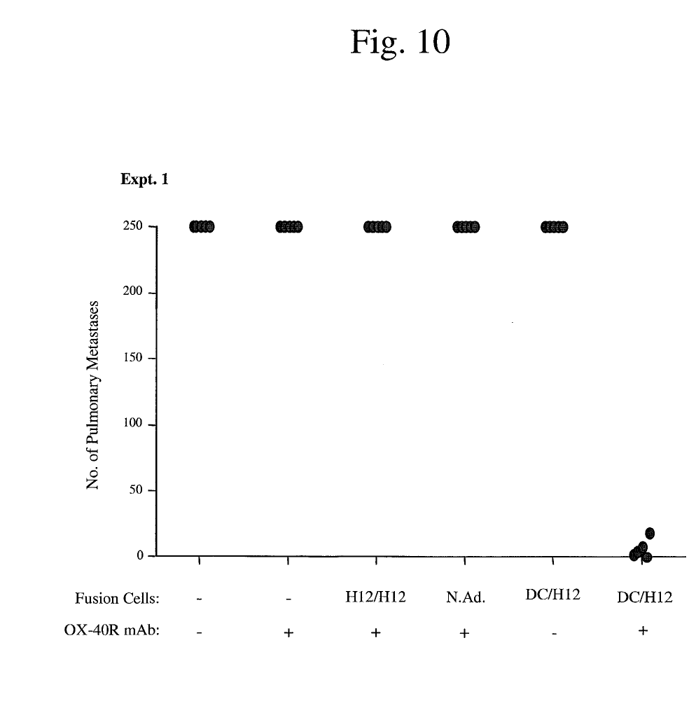

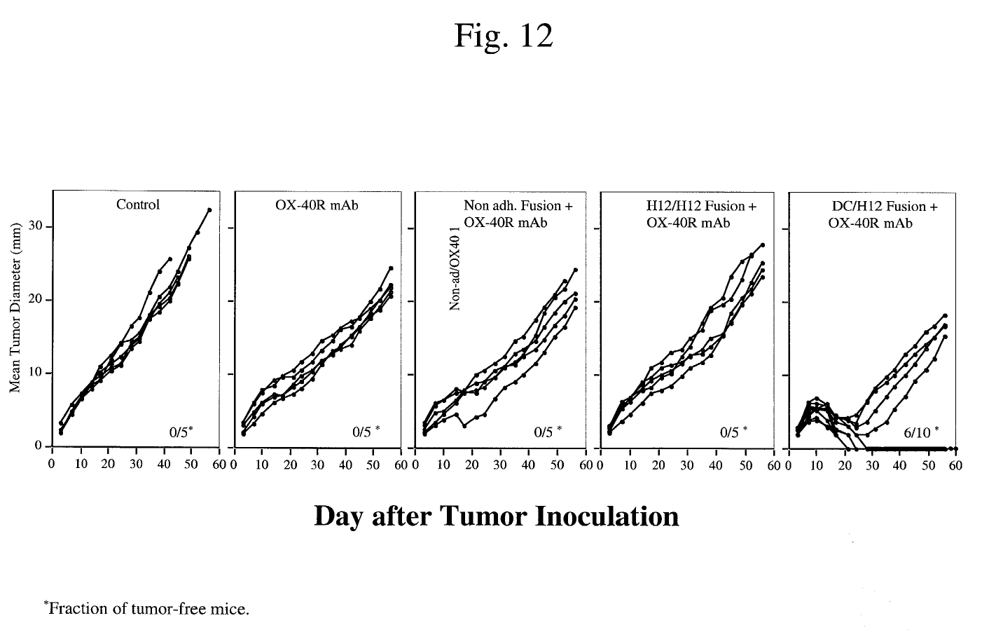

The protocol of electrofusion of DC and tumor cells for treatment of other cancers such as breast carcinoma, renal cell carcinoma, lung cancer, colon cancer, to name just a few, is now possible. In addition to successfully fusing human DC and tumor cells, studies with animal tumor models demonstrated the superb ability of fusion cells to stimulate interferon production by specific tumor immune T lymphocytes. In this treatment setting, mice with a solid tumor (5×7 mm) were successfully treated by one immunization with fusion cells. As illustrated in FIGS. 10, 12 & 13, the therapeutic effects are systemic. Tumors established in the lung, brain, were subjected to the same successful vaccination treatment.

The use of DC-tumor fusion cells may greatly enhance T cell sensitization in the spleen or lymph nodes. The present invention preferably does not employ fusagenic sentai virus or polyethylene glycol (PEG). The related arts use of the items result in very low fusion rates (<3%) which often require selection and long-term culture (e.g. 10 days) to generate sufficient number of cells for possible clinical use. This manipulation could result in diminished function of fused cells. Also, irradiation of fusion cells to prevent tumor growth may damage the antigen-presentation ability of these fusion cells. Our procedures, in contrast using irradiated tumor cells before fusion, thus avoids irradiation of DC or fusion cells to preserve their biological functions.

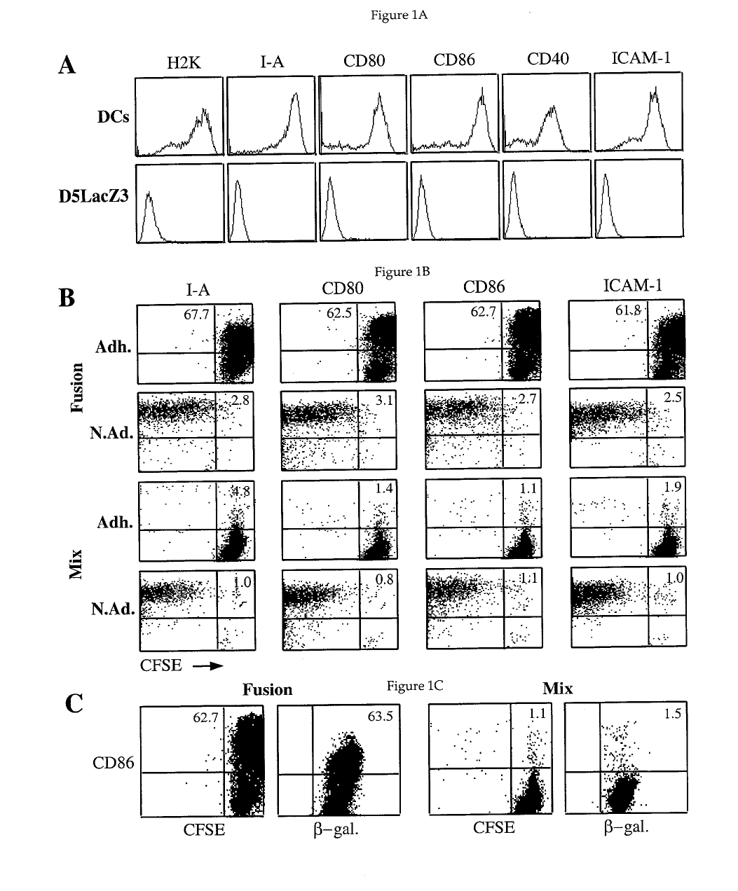

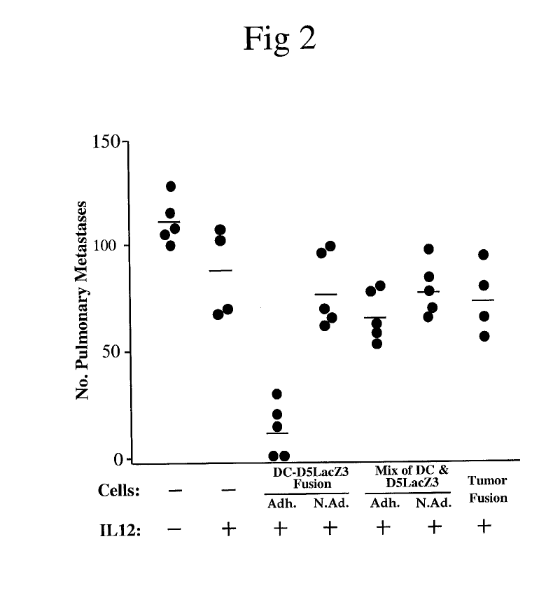

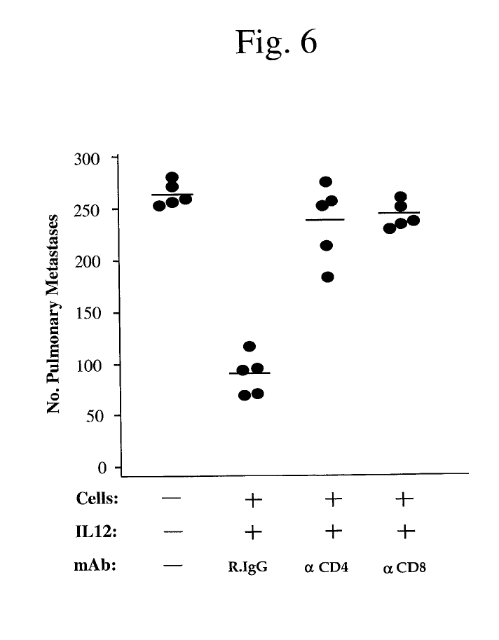

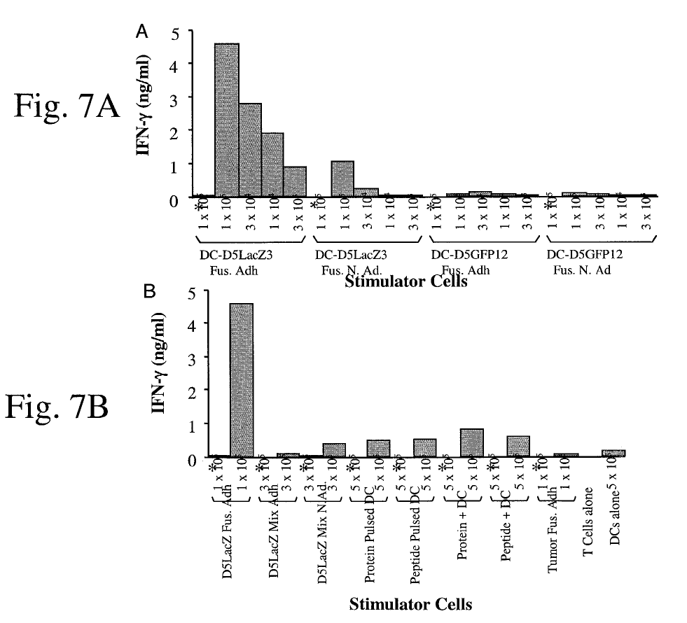

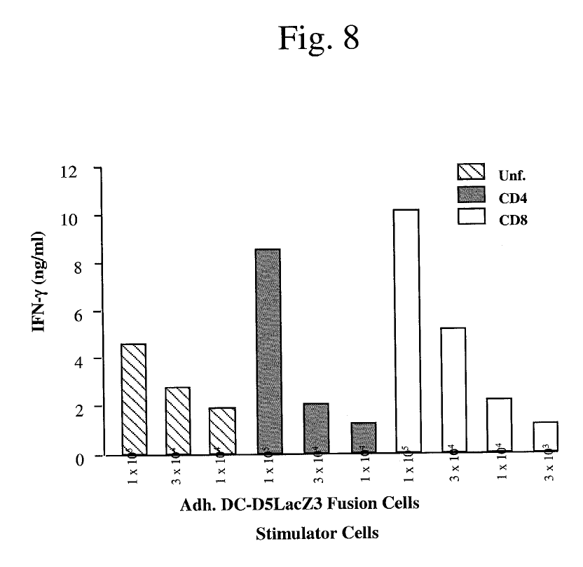

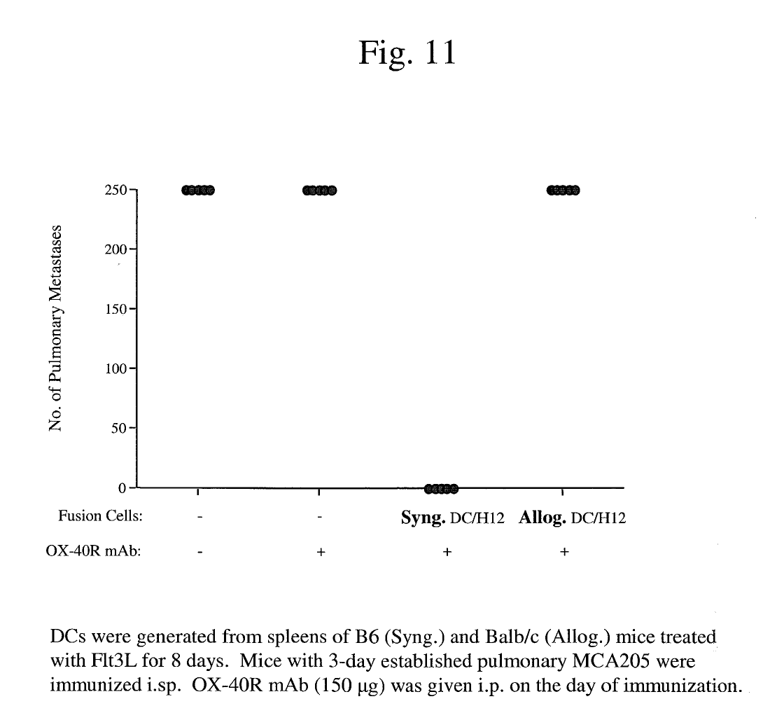

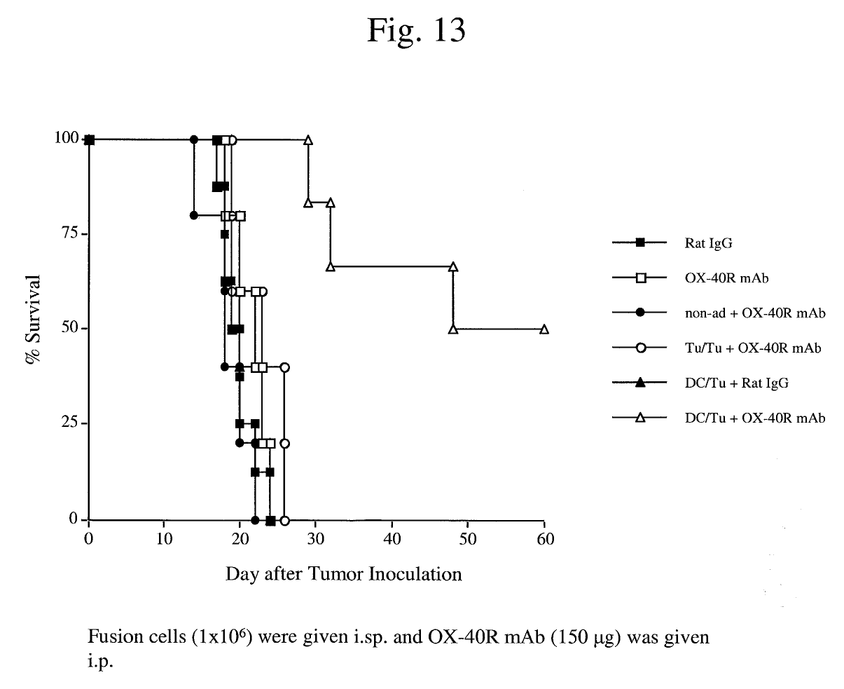

Our results indicate that vaccination with fusion cells was therapeutic in several murine tumor models. In the current study, we confirmed and further demonstrated the success of large-scale fusion of DCs and tumor cells. In most experiments, as many as 300×106 cells could be processed at one time with consistent high fusion rates. As shown in FIG. 1, using a non-immunogenic tumor cell line of B16 melanoma transduced with the LacZ gene, β-gal provided a surrogate tumor rejection Ag for detailed biological and immunological analyses. As shown in FIG. 6, the fusion of mature DCs and tumor cells generated highly immunogenic hybrid cells capable of stimulating both CD4 and CD8 T cells in vitro and in vivo. Active immunotherapy was successful by a single vaccination of mice bearing 3-day established tumors as shown in FIGS. 2, 3 and 10-13. The ability of DC-tumor fusion hybrids to elicit Th1 type immune responses as shown in FIGS. 7 and 8, made them particularly valuable for therapeutic vaccine development for the treatment of cancer. FIG. 8 illustrates that DC-D5acZ3 fusion rate was 60%

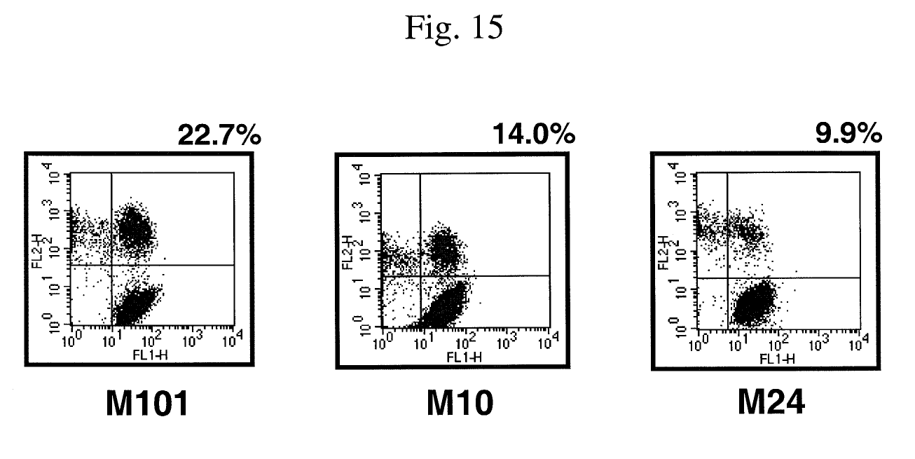

The present invention provides electrofused DC-tumor cell hybrids for activating anti-tumor responses. Although the specific procedures and methods described herein are exemplified using particular cell lines and isolated DCs, they are merely illustrative for the practice of the invention. Analogous procedures and techniques are applicable for the treatment of human subjects, as shown in FIGS. 15-17, and as thereafter exemplified using a human cell line and blood-derived DCs. Therefore, DC-tumor cell hybrids could be used to immunize human patients against their cancer.

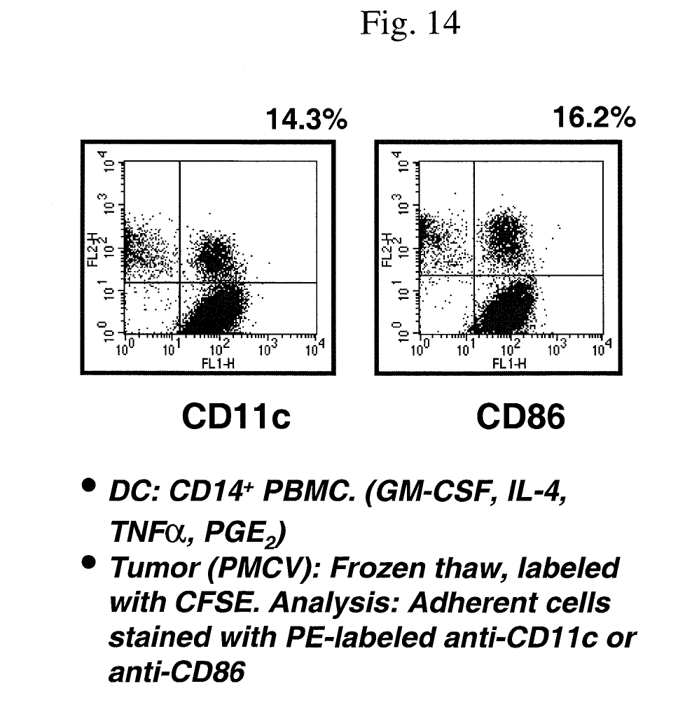

In a preferred embodiment, a sample is provided of the tumor against which an immune response is needed. Such a sample can be obtained when the primary tumor and/or its metastases are removed by surgery, as practiced for example for cancers of the breast, prostate, colon, and skin. When the treatment of the cancer involves chemotherapy and/or radiotherapy rather than surgery, as practiced for example for small cell lung cancer, lymphomas and leukemias, a sample of the tumor can be obtained from a metastatic site, either before treatment or after relapse. Examples of easily-accessible tumor sampling sites are the peripheral blood, bone marrow, peritoneal and pleural effusions, lymph nodes and skin. Alternatively, selected allogeneic tumors of the same hislogy may also be used for fusion provided that the cross-reacting Ags are present. An example of this approach is illustrated in FIGS. 14-16 and Tables 1 and 2.

Tumor cells can be separated from solid tissue samples, using a combination of physical, enzymatic and immunological methods. Macroscopic peritumoral stromal tissue can be removed by dissection prior to reduction of the tumor to a cell suspension. Density centrifugations and antibody-mediated separations can then be performed on the cell suspension as described above. Many fresh tumors can be cultured in vitro to establish tumor cell lines.

The purified tumor cells are then prepared for cell fusion. Three types of tumor partners can be prepared: (i) tumor cells purified from a surgical specimen, (ii) short term established tumor cell lines (iii) allogeneic tumor cells which share antigens with autologous tumor. Short term cultured cells are purified tumor cells which have been cultured for a limited period of time in the presence of appropriate media and growth factors.

A sample is provided with a source of DCs. Such samples include for example peripheral blood and bone marrow cells,; they may be taken from the patient or from a healthy, HLA-compatible donor. From there, two alternatives are available. Functionally-competent DCs can be purified directly from these samples, using various methods described in the literatureor can be generated after in vitro differentiation of the precursors contained in these samples, which can be done by culturing the latter in the presence of cytokines, as described hereunder.

The DC-tumor cell hybrids are generated by electrofusion. Their therapeutic potential is linked to the retention of pertinent DC characteristics and of pertinent tumor cell characteristics. A simple and effective way to enrich fusion cells is by their ability to adhere to a plastic surface. If desired, this procedure allows the elimination of unfused DCs. Pertinent DC characteristics include DC morphology, DC surface markers, DC genetic markers and the capacity to activate immune cells in vitro. At least one of these DC characteristics may suffice to qualify hybrids.

Herein, the term “anti-tumor response in vivo” refers to the in vivo induction of immune effectors that confer resistance to a subsequent challenge with tumor cells, and contribute to the rejection of pre-existing tumor cells. In human subjects, appropriate non-invasive measures can be used for demonstrating the presence of anti-tumor immune effectors. However, the clinical course of the tumor, monitored by imaging techniques and the survival of the patient, will be the prime criterion for the evaluation of the immunotherapy.

Herein, the term “anti-tumor response in vitro” refers to the in vitro activation of autologous immune cells into anti-tumor immune effectors. The latter will contribute to the rejection of the pre-existing tumor cells when infused into the patient.

Herein, the term “DC characteristics” shared by the hybrid of the invention refers to DC morphology, the expression of DC surface markers, the expression of DC genetic markers and the ability for activation of immune cells.

Herein, the term “DC morphology” refers to a typical image observed by scanning electron microscopy. The images of the DC-tumor cell hybrid are compared to those of the parent tumor cell and DC.

Herein, the term “activation of immune cells in vivo” refers to the immune rejection of a residual tumor, as measured by its reduction in size and by the survival of the patient. In vitro correlates of this in vivo state of immunity include for example the detection of blood or tissue immune cells able to react or kill the patient's own tumor cells in vitro. In experimental animals, the quoted expression also refers to the immune resistance to a subsequent inoculation of tumor cells, and to the presence of tumor specific immune effector T cells in the lymphoid organs of the tumor-resistant animals.

Herein, the term “activation of immune cells in vitro” refers for example to a mixed lymphocyte-tumor cell reaction, wherein the dendritic cell/tumor cell hybrid (“the tumor cell”) stimulates one of the following reactions by T-cells (“the lymphocyte”): (1) T-cell proliferation, as measured by tritiated thymidine incorporation; (2) T-cell secretion of cytokines including for example tumor neurosis factor (TNF)α, interferon-gamma (INF-γ) and others, as measured by ELISA, bioassay, or reverse transcription polymerase chain reaction; (3) T-cell-mediated tumor cell lysis, as measured by chromium release or similar assays. This term may also refer to the activation of other immune cells, like monocytes and natural killer cells, and can be measured, for example, by cytokine release or cytotoxic cell assays.

Large-scale preparation of hybrid cells from DCs and tumor cells by the electrofusion technique is a particular advantage of the present invention. Because DCs have multiple veiled processes and dendrites, fusion of them to tumor cells requires specifically designed fusion medium as well as using specific electric field strengths to bring cells in close contact before induction of reversible cell membrane breakdown. Because clinical use of fused cells requires large-volume preparations and immediate use for vaccinations, the present invention has widespread potential applications.

Initially both the tumor cells and the dendritic cells must be isolated and electrofused. The tumor cells can be obtained from purification from a surgical specimen, short term cultured established tumor cell line, or allogeneic tumor cells which share antigens with the autologous tumor. The tissue is generally digested with collagenase and a suspension is purified from surgical material or from tissue culture.

It is also necessary to generate human dendritic cells. Monocytes are isolated from peripheral blood mononuclear cells (PBMC) obtained from leukaphoresis. Media containing Granular Monocytes-Colony Stimulating Factor (GM-CSF) and interleukin-4 (IL-4) are used for DC culture for seven days. During the last two days of the culture period, TNF-α and PGE₂ are added. The recovered cells are dendritic cells that show a mature phenotype and express IL-12.

The next step is to create fusion between the dendritic cells and tumor cells. The fusion media that may be used are of several types. One type of fusion media (Media A) is created by combining 5% glucose with 0.1 mM calcium acetate, 0.5 mM magnesium acetate, and 1% bovine BSA adjusted to a pH of 7.2. The pH can be adjusted by using histidine. An alternative type of media that can be used (Media B) is created by combining 5% glucose with 0.1 mM calcium acetate and 0.5 mM magnesium acetate without BSA.

For the fusion process, the ratio of dendritic cells to fusion cells should be about 1 to 2:1. For example, a 5 cc amount of fusion media would compel up to 75 million cells, with 50 million cells being dendritic cells and 25 million cells being tumor cells. The cells are aligned (dielectrophoresis) with alternative current at about 150 V/cm for 10 seconds. This is immediately followed by exposure of the cells to direct current of 1200V/cm for 25 μ seconds (i.e., a high voltage for a short period). The direct current voltage is sometimes reduced by up to 20% depending on the tumor cell line. The cells are then diluted 1:10 in complete RPMI 1640 media with 10% human AB serum. All of the cells are cultured in a flask with RPMI 1640 medium containing 10% FCS overnight (e.g.,12-18 hours). After the culture period, the flask is washed to remove any non-adherent cells (i.e., those cells not adhering to the walls of the flask). Adherent cells are those that compel DC-tumor fusion cells and, non-adherent cells are discarded because they do not compel DC-tumor cells, i.e., the non-adherent cells are mostly not fused and fused DCs. The adherent cells can be harvested by trypsin. Specific examples including materials and methods are provided below. It is to be understood that these examples are provided for illustrative purposes only.

### Methods and Materials

Mice: Female C57BL/6N (B6) mice were purchased from the Biologic Testing Branch, Frederick Cancer Research and Development Center, National Cancer Institute (Frederick, Md.). The mice were maintained in microisolator cages under specific pathogen-free conditions. All mice were used at 8-12 wks of age.

Tumors: The B6 derived B16.F10.BL6 melanoma has been previously described. A cloned cell line, D5 from the melanoma was transduced with the LacZ gene to express β-gal by a previously described method. D5acZ3 was a cloned β-gal expressing cell line. As a control, we also transduced the D5 melanoma with plasmid gene encoding green fluorescence protein (GFP) by the identical method. D5GFP12 was a cloned cell line with consistent and high GFP expression. These melanoma cell lines were maintained at 37° C. in RPMI 1640 medium supplemented with 10% heat-inactivated FCS, L-glutamine and antibiotics (complete medium or CM) as previously described. Murine GL261 glioma and MCA205 (H12) sarcoma are similarly maintained in vitro.

DC Preparation: DCs were generated from spleens of mice that had received i.p. injections of 5-10 μg F1t3 ligand (Immunex Corp., Seattle, Wash.) for 8-10 days (23). Single cell suspensions of the spleens were enriched for CD11c cells by positive selection using MACS CD11c MicroBeads (Miltenyi Biotec, Auburn, Calif.) following manufacturer's instructions. These cells were cultured in CM containing GM-CSF (10 ng/ml) and IL-4 (10 ng/ml) for 1-2 days to allow DC maturation. As shown in FIG. 1A, the DC preparations contained approximately 80% cells showing a phenotype of mature DCs with high expression of I-A, CD80, CD86 and CD40.

Electrofusion of DCs and tumor cells: DCs and irradiated (5000 cGy) tumor cells were mixed and suspended in 280 mM glucose or sorbitol solution with 0.1 mM Ca (CH₃COO)₂, 0.5 mM Mg (CH₃COO)₂ and 0.3% bovine serum albumin at 2:1 ratio. The pH of the fusion medium was adjusted to 7.2-7.4 with L-histidine (all chemicals were from Sigma). The medium was isoosmotic (300 mOsm) with a specific resistivity of about 400 kΩcm. After centrifugation, the cells were resuspended in the same fusion medium in the absence of bovine serum albumin. Routinely, 5 ml of cell suspension containing 75×10⁶ cells were processed using a specially designed concentric fusion chamber. For electrofusion, a pulse generator (model ECM 2001, BTX Instrument, Genetronics, San Diego, Calif.) was used for application of field pulses. Electrofusion involves two independent but consecutive steps. The first reaction is to bring cells in close contact by dielectrophoresis, which can be accomplished by exposing cells to an alternating (ac) electric field of relatively low strength. Cell fusion can then be triggered by applying a single square wave pulse to induce reversible cell membrane breakdown in the zone of membrane contact. For the current study, fusion was accomplished by dielectrophoresis with an ac pulse of 240 V/cm for 10-25 s followed by a direct current (dc) pulse of 1200 V/cm for 25 μs. The fusion mixture was allowed to stand for 5 mm before suspending in CM and incubated at 37° C. overnight. The fusion cells were harvested based on cell adherence. The nonadherent (N.Ad.) cells consisted of mainly DCs and the adherent (Adh.) cells, mainly fusion hybrids and tumor cells. To determine fusion efficiency, tumor cells were pre-labeled with the intracellular green fluorescent dye, CFSE and after fusion, heterokaryons were detected by staining with PE-conjugated niAbs against cell surface markers expressed only by DCs. Typically, fusion hybrid cells were double-positive upon FACS analysis. To confirm fusion efficiency, confocal microscopy demonstrating individual cells of dual fluorescence and Giemza-stained cytocentrifuge preparations for evidence of multinucleated cells were also performed.

In vitro assays: Specific tumor-immune T cells were generated from tumor-draining lymph nodes (LNs) as described previously. Briefly, B6 mice were inoculated s.c. with 1.5×10⁶ D5acZ3 cells on the flank. Nine days later, draining inguinal LNs were harvested and actiyated in vitro with anti-CD3 mAb and IL-2 for 5 days. Previous studies have demonstrated that these activated tumor-draining LN cells contained both CD4 and CD8 tumor-specific T cells and the adoptive transfer of these cells mediated potent antitumor effects against established tumors. In the current study, these activated immune cells were used for analyses of stimulatory activities of DC-tumor fusion hybrids for cytokine secretion. Routinely, 2×10⁶ activated tumor-draining LN T cells were stimulated with graded doses of DC-tumor fusion cells in CM. Twenty-four h supernatants were collected and IFN-γ concentrations were determined by ELISA using paired mAbs (BD PharMingen, San Diego, Calif.). Additional stimulator cells included DCs pulsed with β-gal protein (40 μg/10⁶ cells/ml) or the H-2k^{b}-restricted peptide, DAPIYTNV (10 μg/10⁶ cells/ml) for 12 and 4 h, respectively. Activated tumor-draining LN cells were also stimulated with DCs in the presence of β-gal protein (40 μg/ml) or peptide (10 μg/ml). In some analyses, immune T cells were enriched for CD4 and CD8 T cells by positive selection with anti-CD4 and anti-CD8 labeled MicroBeads (Miltenyi) before DC-tumor fusion cell stimulation.

FACS analysis: Tumor cells were labeled with CFSE (Molecular Probes, Inc., Eugene, Oreg.) at a concentration of 10×10⁶/Ml in the presence of 5 μM dye for 10 min at 37° C. Labeling was terminated by adding ice-cold HBSS. PE-labeled mAbs against DC markers including CD11c, CD40, CD80, CD86 and mAbs against I-A and ICAM-1 were purchased from BD PharMingen. Analyses of 10,000 cells for each sample were performed using the FACS Calibur (Becton Dickinson, Mountain View, Calif.).

To analyze β-gal expression by fusion hybrid cells, fluorescein di-β-D-galactopyranoside (FDG; Molecular Probes, Inc.) staining was performed following the manufacturer's suggested procedure. Briefly, fusion cells were stained with the PE-conjugated anti-CD86 and resuspended at 10>10⁶/ml. One million cells suspended in 100 μl of PBS containing 5% FCS were incubated with 100 μl of 2 mM FDG (20 mM in 10% dimethyl sulfoxide/10% EtOH/dH₂O) for 1 min at 37° C. The staining was stopped with the addition of an excess of ice-cold PBS with 5% FCS. Cells were analyzed by the FACS as described above. In this system, electrofusion was performed with unlabeled tumor cells.

Active immunotherapy: Eight to 12-wk-old B6 mice were inoculated i.v. with 3×10⁵ D5acZ3 cells suspended in 1.0 ml of HBSS through the tail vein to establish pulmonary metastases. Three days later, tumor-bearing mice were vaccinated with DC-tumor Adh. fusion cells (0.3×10⁶) suspended in 10 μl of HBSS through intranodal injections at both inguinal LNs as described (27). Treated mice were also given 0.2 μg of IL-12 (a gift from Genetics Institute, Cambridge, Mass.) in 0.5 ml of HBSS i.p. for 4 days as an adjuvant. Mice with pulmonary metastases were sacrificed on day 17 or 18 and metastatic nodules on the surface of the lung were counted. In some experiments, survival of the treated mice was monitored and recorded as the endpoint of immunotherapy. To study the role of CD4 and CD8 T cells in response to DC-tumor fusion cell vaccination, mice were given i.v. injections of the anti-CD4 (anti-L3T4, clone GK1.5) or anti-CD8 (anti-Lyt2.2, clone 2.43) mAb ascites (0.2 ml diluted to 1.0 ml with HBSS) on days 1 and 7 of tumor inoculation to deplete respective T cell subsets. The effectiveness of T cell depletion was confirmed on the day of lung harvest (day 17) by FACS analyses of spleen cells from mAb-treated mice.

Statistics analysis: The significance of differences in numbers of pulmonary metastatic nodules was analyzed by the Wilcoxon rank sum test. A two-tailed p value of ≦0.05 was considered significant.

We have provided conclusive evidence of electrofused DC-tumor cells. In “Clinical Immunology”, Vol. 104, No. 1, pp. 14-20 (2002), which is incorporated herein in its entirety by reference thereto, illustrated multinuclear fusion hybrids with confocal fluorescent micrographs. Additional results are described herein.

Characteristics of DC-D5acZ3 fusion hybrid cells: FIG. 1 illustrates FACS profiles of hybrid cells generated by electrofusion in accordance with the present invention. FIG. 1A illustrates the Phenotypes of DCs and D5acZ3 tumor cells. FIG. 1B, FACS analyses of electrofusion of DCs and D5acZ3 cells. Tumor cells were stained with CFSE (green) prior to fusion. After overnight culture, both adherent (Adh.) and nonadherent (N.Ad.) cells were stained with PE-conjugated mAbs as indicated. FIG. 1C, Expression of β-gal in fusion cells was detected by direct immunofluorescence staining with FDG. In this case, tumor cells were not stained before fusion and after fusion, Adh. cells were analyzed. For comparison, CFSE-labeled tumor cells were also used for fusion to estimate fusion efficiency. Numbers in FACS dot plots are percentages of double-colored fusion cells. FACS analyses revealed that DCs we prepared displayed a characteristic phenotype of mature cells with the expression of MHC class I and II, costimulatory molecules and ICAM-1 while the tumor cells lacked all these molecules on their cell surface as shown in FIG. 1A. Electrofusion of these two cells resulted in the generation of heterokaryons that expressed both green fluorescence (CFSE) of tumor cells and a number of DC markers as shown in FIG. 1B. In over 30 experiments, fusion efficiency was consistently greater than 30%. The majority of the double-stained fusion cells was detected in the Adh. fraction of fusion preparations while the N.Ad. cells contained either unfused or fused DCs. The high percentages (>61%) of fusion as shown in FIG. 1B, were the result of electrofusion because the Adh. fraction of a mixture of DCs and tumor cells did not yield a significant number of double-positive fusion cells. As shown in FIG. 1C, the fusion hybrids also retained the surrogate tumor Ag as revealed by β-gal staining. To demonstrate β-gal expression, fusion was carried out using nonlabeled tumor cells. Fusion with CFSE-labeled tumor cells served to establish the fusion rate. Analysis was done on Adh. cells of both fusion and the mixed cell control preparations.

Although essential, FACS analyses might give false results of positive fusion because aggregated cells or unfused DCs which had ingested tumor debris could also appear to be double-positive. To confirm the occurrence of fusion, confocal microscopy was employed to demonstrate the existence of dual fluorescence on individual cells. Furthermore, fusion hybrids were visualized on cytocentriftige preparations as multinuclear cells. These results provide an unequivocal evidence for verification of fusion.

Immunological requirements and characteristics of immunotherapy with DC-tumor/fusion hybrid cells: In a previous publication, we demonstrated that vaccination with fusion cells had therapeutic effects. However, it was not clearly defined whether DC-tumor fusion were the only cells mediating antitumor effects. In the experiment illustrated in FIG. 2, mice with 3-day established pulmonary tumor metastases were vaccinated with DC-D5acZ3 fusion cells (0.3×10⁶, 42% fusion rate) at both inguinal LNs. IL-12 (0.2 μg in 0.5 ml HBSS with 0.1% mouse serum) was administered i.p. for 4 days. Horizontal bars represent average number of metastatic nodules. The numbers of metastases in mice treated with Adh. cells of DC-D5acZ3 fusion and IL-12 were significantly reduced as compared to all other groups. As shown in FIG. 2, active immunotherapy with DC-D5acZ3 fusion cells and IL-12 significantly reduced the numbers of pulmonary D5acZ3 metastases. In FIG. 2 vaccination with tumor-tumor fusion preparation was ineffective. In addition, neither N.Ad. cells from electrofusion nor cells from not fused mixture of DCs and D5acZ3 tumor cells demonstrated antitumor effects. It was therefore concluded that fusion of DCs and tumor cells resulted in the generation of highly immunogenic hybrid cells that a single vaccination was capable of mediating the regression of established tumors.

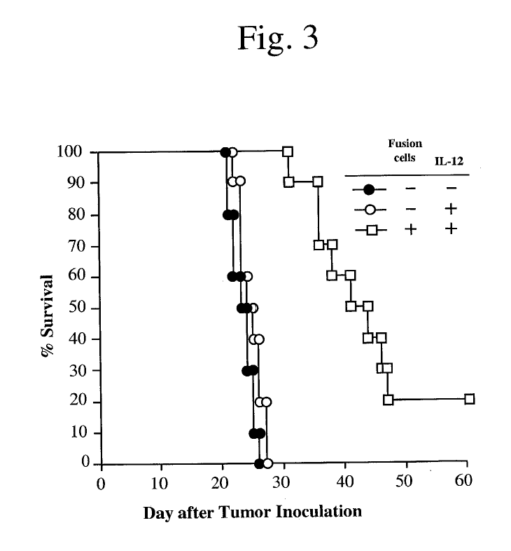

One of the concerns for evaluating antitumor effects by enumerating metastases on the surface of the lung had been whether the reduction of metastastic nodules was a meaningful measure for therapeutic benefits. We therefore performed an independent experiment where the survival of treated mice was used as the endpoint. The experimental procedure depicted in FIG. 3 is the same as that in FIG. 2, except survival was recorded as the endpoint and each group consisted of ten mice. Mice without treatment or treated with IL-12 alone succumbed to the growing tumors with similar median survival time (24-25 days). In contrast, mice vaccinated with DC-LacZ3 fusion cells plus IL-12 demonstrated a prolonged median survival of 40 days and two of 10 treated mice were apparently cured of the tumor. Therefore, reduction of pulmonary metastases was correlated with the survival of treated mice.

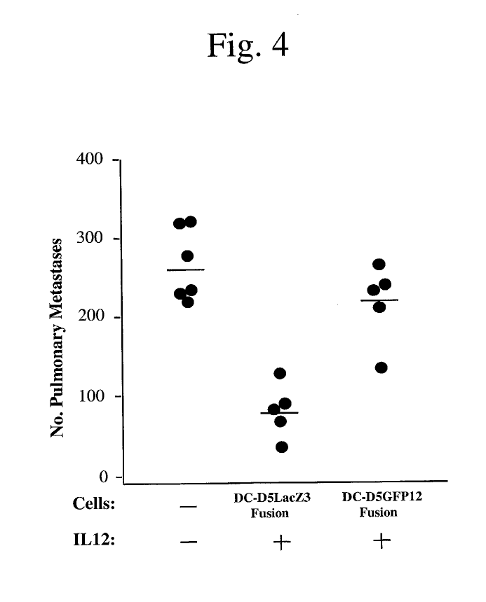

The specificity of immunotherapy was first demonstrated by the failure of DC-D5acZ3 fusion cell vaccination to mediate the regression of the control D5GFP12 tumor. The specificity was further evaluated by examining the ability of DC-D5GFP12 fusion cells to affect the progression of D5acZ3 metastases. FIG. 4 illustrates the specificity of active immunotherapy mediated by vaccination with DC-tumor fusion hybrid cells and IL-12. Although treatment of D5acZ3 tumors is effective with vaccination by DC-D5acZ3 fusion hybrids, the treatment is not effective against an irrelevant tumor, D5GFP12. The experiment procedure is the same as that described in FIG. 3. Mice bearing 3-day established pulmonary D5acZ3 metastases were treated with either DC-D5acZ3 or DC-D5GFP12 fusion hybrid cells plus L-12. Fusion rates for DC-D5acZ3 and DC-D5GFP12 were 51% and 50%, respectively. No significant therapeutic effects were seen when D5acZ3 tumor was treated with fusion cells generated from D5GFP12 tumor cells. Results in FIG. 4 clearly demonstrated the failure of GFP fusion cells to mediate tumor regression while significant reduction was observed if vaccine fusion cells were generated from β-gal expressing D5acZ3 tumor cells. Thus, active immunotherapy requires the specific tumor cells for generating fusion cells.

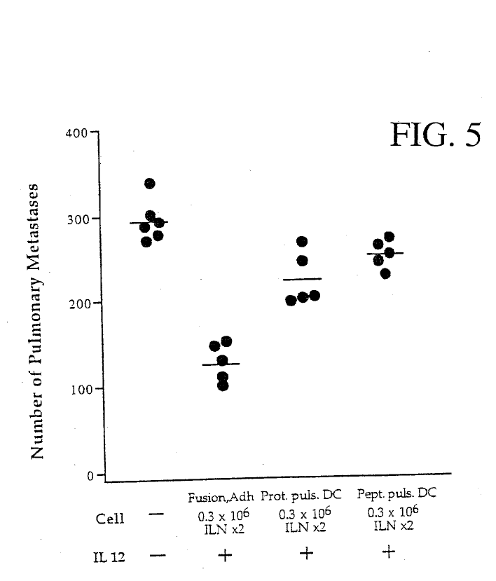

FIG. 5 illustrates that the therapeutic efficacy is mediated by vaccination with fusion cells,but not with protein or peptide-loaded DCs.

DC-tumor fusion hybrids expressed both MHC class I and II molecules on their surface. They should be equipped to initiate both CD4 and CD8 T cell immune responses. We therefore analyzed the effector T cells responsible for antitumor activity. Mice were twice administered with mAbs against CD4 or CD8 T cells before and after vaccination with fusion cells. Depletion of the respective cell population by >80% was confirmed by the FACS analysis (data not shown). FIG. 6 illustrates the requirement of both CD4 and CD8 T cell responses for effective fusion cell vaccine therapy. Depletion of CD4 and CD8 cells was accomplished by the administration of corresponding monoclonal antibodies and confirmed by FACS analysis of spleen cells from treated animals. The experimental procedure is the same as that in FIG. 2 except in some groups, mice were also depleted of CD4 or CD8 T cells by the administration of mAbs. The fusion rate was 49%. The only group of mice demonstrating significant antitumor effects was that treated with control Ab (Rat IgG). The finding that depletion of either CD4 or CD8 T cells in vivo abrogated the antitumor effects was consistent with the conclusion that both MHC class I- and II-restricted CD8 and CD4 immune responses were associated with the therapeutic effects of fusion cell immunotherapy.

In vitro T cell stimulatory activity of DC-tumor fusion hybrid cells: DCs generated in vivo as a result of F1t3 ligand administration displayed heterogeneous subpopulations of functionally distinct cells. Mature DCs have the potential to induce either Th1 or Th2 CD4 T cell responses. Similarly, CD8 T cell responses also demonstrated divergent type 1 or 2 responses. In general, type 1 response is exemplified by the ability to produce Th1-associated cytokines such as IL-2, TNFα and IFN-γ while type 2 response is associated with IL-4, IL-5, IL-6, IL-10 secretion. It is evident that type 1 responses are likely the optimal immune response that effectively mediate tumor regression. We therefore examined the ability of fusion cells to stimulate cytokine secretion by immune T cells. In a dose titration experiment, we found that large amounts of IFN-γ were produced by T cells upon stimulation with fusion cells. FIG. 7 illustrates IFN-γ secretion by activated tumor-draining LN T cells after stimulation in vitro with electrofusion cells. IFN-γ concentrations were determined by the ELISA 24 hours after stimulation with fusion cells and cell preparations as indicated. D5acZ3 immune cells secreted IFN-γ after stimulation with DC-D5acZ3 fusion cells. All other cell preparations stimulated minimum or no IFN-γ production. Twenty-four h supernatants were analyzed by ELISA. Fusion rates for D5acZ3 and D5GFP12 were 60% and 62%, respectively. Asterisks (*) indicate stimulator cells alone. DC loading with β-gal protein or peptide was carried out as described. As few a 3×10³ fusion cells containing approximately 60% fusion hybrids stimulated the secretion of 1000 pg/ml of IFN-γ from 2×10⁶ immune T cells as shown in FIG. 7. This reaction was immunologically specific because fusion cells prepared from DCs and the D5GFP12 tumor failed to stimulate IFN-γ secretion from D5acZ3-immune T cells. In the IFNγ ELISA, we also examined the T cell stimulatory activities of other forms of DC loading including DCs pulsed and incubated with β-gal protein as well as with the H-2 K^{b}-restricted β-gal peptide, DAPIYTNV. As indicated in FIG. 7B, DC-tumor fusion cells were far superior to all other DC-loading preparations in stimulating IFN-γ secretion from immune T cells. Furthermore, fusion of tumor cells in the absence of DCs did not have any activity. Consistent with the characteristics of fusion cells, DC-tumor hybrids were capable of stimulating IFN-γ secretion from both CD4 and CD8 immune T cells. FIG. 8 illustrates analyses of IFN-γ secretion by purified CD4 or CD8 T cells from activated tumor-draining LN cells. ELISA is the same as that described in FIG. 7. As illustrated in FIG. 8, DC-D5acZ3 fusion rate was 60%.

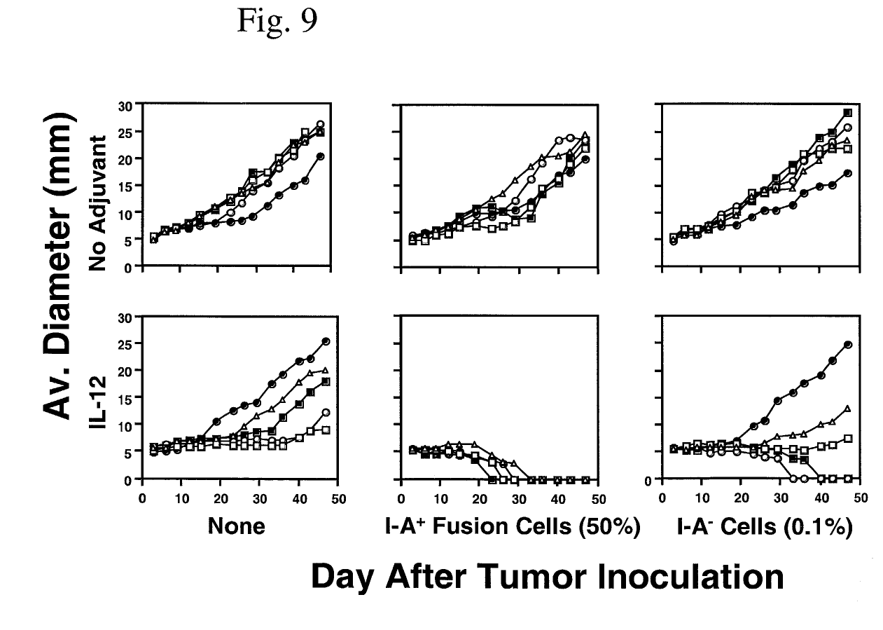

Although we used the D5acZ3 tumor as an example for demonstrating the immunogenicity and therapeutic efficacy of DC-tumor electrofusion hybrid cells as shown in FIGS. 9-13, other animal tumors including GL261 glioma and MCA205 sarcoma would respond to the therapeutic effects of DC-tumor fusion cells. These additional tumor models demonstrate the superb therapeutic effects of fusion cells against naturally occurring tumor-associated Ags because no genetic modification of these tumor cells was done in these experiments.

We also have significant experimental data to illustrate successful fusion of human DCs and melanoma cells as shown in FIGS. 14-16. In these cases, human DCs were generated from peripheral blood mononuclear cells (PBMC) obtained from leukaphoresis of both cancer patients and healthy human volunteers. Routinely, adherent monocytes (CD14⁺ cells) were allowed to adhere onto plastic culture flasks for 24 hours in serum-free X-vivo 20 medium (BioWhittaker). The adherent cells were cultured in RPMI 1640 medium with 10% heat-inactivated FCS supplemented with human GM-CSF (10 ng/ml) and IL-4 (10 ng/ml) for 7 days. During the last 2 days of culture, DCs were matured by adding TNFA (10 ng/ml) and PGE₂ (1 μg/ml). DCs generated in this culture condition express high levels of MHC class II, CD11c, CD80, CD86, CD40, and CD83 molecules with greater than 80% homogeneity.

For electrofusion, autologous DCs and melanoma cells at a ratio of 1:1 are mixed and washed, resuspended in 0.3 M glucose and transferred into a fusion chamber. Fusion will be accomplished by first aligning the cells to form close cell-to-cell contact at 150 V/cm for 10-15 seconds. Alignment may be optimized empirically by observation under a microscope. This provides the opportunity for immediate modification of parameters used if problems are observed. Immediately following alignment, a pulse with a direct current of high intensity and short duration (1200 V/cm, 25 μ seconds) will be applied to induce reversible cell membrane breakdown. Cells apposed during electric membrane breakdown will fuse spontaneously during the resealing phase, resulting in membrane continuity of the involved cells. Generally, the entire process requires 30 minutes to 3 hours in order for complete cell fusion to occur. The fusion mixture will be cultured in CM in tissue culture flasks for 1 day. Unfused DCs can be rinsed out because most fused cells will adhere to the plastic surface. Our own experience indicates that the procedure we use will allow the generation of DC-tumor hybrids in the fusion rate of at least 10% (in most experiments, a fusion rate of greater than 15% is observed) as shown in FIGS. 14-16. The above outlined procedure is a general description and many parameters may be moderated empirically for maximum fusion for different tumor cells.

To determine fusion efficiency in preliminary experiments, tumor cells were stained with a green fluorescence dye, 5-(and -6)-carboxyfluorescein diacetate, succinimidyl ester (CFDA; Molecular Probes, Eugene, Oreg.) before fusion. Staining with CFDA will not affect cell viability and function in vitro and in vivo. After fusion, the adherent cells may be stained with PE-conjugated antibodies against cell surface molecules present on DCs, but not on tumor cells such as MHC class II, CD80, CD86 or CD11c. Fused hybrids will be detected in the double-positive fraction upon analysis by flow cytometry. Because the adherent fusion fraction of cells contains very few DCs (˜15%), cells express CD80, CD86 or CD11c are likely to be fusion hybrids. The estimate fusion rate may be calculated by subtracting the percentage of CD80, CD86 or CD11c positive cells in control DC-tumor mix from that in the fusion population.

The minimum specification for the apparatus is that it has both low-voltage alignment capacities and the ability to generate a high-voltage fusion pulse. Initially, we found BTX 2001 ECM™ (BTX, Inc., San Diego, Calif.) used in conjunction with the BTX Optimizer™ to be adequate.

Perhaps, the most important aspect of human DC-tumor fusion hybrid cells is their high immunogenic activity or reactivity in stimulating IFNγ and GM-CSF secretion from well-defined T cell clones or cell lines as shown in Table 1. Further analysis demonstrated that T cell reactivity was induced only by stimulation with fusion products generated from DCs expressing appropriated MHC molecules and tumor cells expressing relevant Ags as shown in Table 2. These results thus demonstrate the feasibility of generating effective DC-tumor fusion cells from allogeneic tumor cells if they express tumor-associated Ags. They also indicate that autologous or allogeneic MHC-matched DCs may be used for electrofusion.

DISCUSSION: Somatic fusion of DCs and tumor cells combines the DC's superior ability of Ag processing and presentation with a rich source of unmodified tumor-associated Ags. This form of Ag presentation is theoretically attractive in vaccine development for cancer immunotherapy. However, as we pointed out, the prevalent technology of fusion using PEG is often plagued by toxicity, low fusion efficiencies and poor reproducibility. With this approach, it is the technical rather than conceptual aspects that hampered progress in laboratory analyses and for clinical application. The significance of the current report lies with the development of a reliable technique for fusion of DCs and tumor cells. As demonstrated, electrofusion of murine DCs and a variety of tumor cells consistently yielded a fusion rate of greater than 20%. The criteria for verifying the existence of heterokaryon fusion hybrid cells have been stringent including FACS analyses, confocal fluorescence microscopy, cytospin preparations as well as DNA content analysis (data not shown). The current system allows large-scale preparations of DC-tumor hybrid cells without additional elaborate procedures for isolation and purification. It is particularly suitable for immunological studies and for clinical use.

Although simple in overall concept, the mechanism of electrofusion is still not fully understood. As a result, improvement of the methodology remains largely on an empirical basis. In some cases, the electrical signal generators as well as fusion chambers may have to be custom built. The required chamber, pulse generator and the biology of the experiment require appropriate interfacing and matching to one another. Two theoretically important manipulations are essential to achieve fusion between two independent cells. First, a tight membrane contact between cells is preferred for fusion. This can be accomplished by exposing cells to an alternating nonuniform electric field of low strength, inducing a process termed “dielectrophoresis”. As a result of Brownian movement and the repellent electrostatic forces arising from the net negative charge on the outer membrane surface, cells in suspension will not normally come into membrane contact. However, when an external electric field is imposed on cells, they become polarized and behave as electric dipoles in the medium. Dipoles are aligned in an electric field and are driven toward or away from areas of high field gradient by the dielectrophoresis force. In this regard, an inhomogeneous electric field will facilitate cell migration because the field intensity is not equal on both sides. If the particles approach during dielectrophoresis they are attracted to each other due to their dipoles. This leads to the formation of “pearl chains” of cells. As a rule, larger cells experience greater force, thus align faster.

The fundamental reaction in electrofusion is reversible membrane breakdown. When short-duration electric impulses applied across cell membrane exceed a critical threshold, membrane will become transiently but highly permeable. The field strength, Ec required to achieve membrane breakdown of a spherical cell can be calculated using the integrated Laplace equation:

Vc=1.5αEc cosθ

where α is the cell radius, θ is the angle between a certain membrane site in relation to the field direction and Vc is the membrane breakdown voltage.

Membrane resealing occurs rapidly and spontaneously after cessation of the breakdown pulse. Application of the breakdown pulse leads to fusion if the membranes of two cells are in close contact which is induced by dielectrophoresis. It is a fortunate accident of nature that the points of contact during dielectrophoresis coincide with the locations on membrane where the pulse causes the greatest membrane breakdown. In the standard electrofusion, cell movement and membrane contact can be visualized under a microscope, thus providing guidelines for monitoring experimental conditions. The complexity of each individual cell system dictates the requirement for understanding the basic physical properties governing the procedure. Therefore, experimental conditions must be tailored for each unique cell type and system. Although it is not possible to provide a protocol which can be used for any cell type, the principal guidelines should allow the investigator to determine which boundary conditions are optimal for the system under study. Our experience indicates that although both murine and human DCs behave consistently, for unknown reasons, different tumor cell lines display different susceptibilities to electrofusion that need to be optimized experimentally. Overall, electrofusion technique has a member of advantages compared to conventional fusion techniques by means of viruses or chemical agents. The greatest advantages are high fusion rates and reproducibility which are prerequisites to any biological system. We have conducted more than 300 fusions with 10 different human and murine tumor cell lines. The minimum fusion rate was 10% and in some cases, as high as 75% was achieved. FIGS. 14 and 15 illustrate electrofusion of human DCs & melanoma cells. Human DC were generated by culturing CD14⁺ monocytes with GM-CSF and IL-4 for 7 days. During the last 2 days of culture, TNF-α and PGE-2 are also added. Human DC showed a mature phenotype. Tumor cells were labeled with CFDA (FL1) before fusion. After electrofusion, cells were cultured overnight and adherent cells were stained with anti-CD86-PE (FL2). Fused cells are double positive for both FL1 and FL2.

Technique development without demonstrable biological significance associated with it, would not be meaningful and eventually become obsolete. In this regard, our results demonstrated that DC-tumor fusion cells stimulated an antitumor immune response capable of eradicating established tumors. The use of DCs as a vehicle to deliver Ags to the immune system is supported by many experiments with primarily mature DCs although uptake of Ags is more efficient by immature DCs. The current approach using electrically fused DC-tumor hybrids also employed mature DCs because Ag uptake is not a prerequisite in our system. In addition, mature DCs are an appropriate choice based on an extended half-life of Ag-presenting MHC molecules. In the approach of loading DCs with tumor peptides, serious concerns have been the varying affinity and off rate of the peptides. By contrast, fusion utilizes viable tumor cells and our preliminary experiments indicated that they survived in vitro for at least 7 days without losing characteristic APC molecules or tumor-associated Ags (data not shown). Although the majority of work used β-gal as a surrogate tumor Ag, as shown in FIGS. 9-13, therapeutic activities of DC-tumor fusion cells have also been demonstrated against undefined natural tumor rejection Ags on the GL261 glioma as well as in the murine MCA 205 sarcoma system.

In vitro and in vivo analyses revealed the ability of DC-tumor fusion hybrids to stimulate both CD4 and CD8 T cells and the activation of both T cell subsets was required for antitumor effects in vivo. In vitro, the fusion cells induced IFN-γ secretion from both CD4 and CD8 T cells in an immunologically specific manner. For the treatment of established tumors, however, DC-tumor cell vaccination alone was not sufficient to mediate therapeutic effect on efficacy. As shown in FIGS. 2, 3 and 9, their function requires the use of IL-12 as an immunoadjuvant. Recently, it has been suggested that in vivo ligation of the costimulator, OX-40 receptor (OX-40R) by mAb enhanced the Th1 response induced by immunization with Ag-pulsed DCs . The results shown in FIGS. 10-13 also indicate that successful active immunotherapy with DC-tumor fusion cells can also be accomplished by the administration of the OX-40 receptor (“OX-40R”) mAb instead of IL-12. OX-40R is a receptor for costimulation which is rapidly expressed after T-cell activation. Similarly, another costimulatory molecule, 41BB, is also up-regulated upon T-cell activation. We recently demonstrated that in vivo ligation of 41BB with mAb has similar adjuvant activities as IL-12 and OX-40R mAb in active immunotheraphy with DC-tumor fusion cells.

In the current study, a single vaccination resulted in the regression of 3-day established tumors. Obviously, the immunization induced a primary antitumor immune response. In many reported cases, therapeutic immunization with DCs loaded with tumor Ags requires repeated administrations. It would, therefore, be important to confirm whether effective booster doses can be effective against large tumors.

Demonstrated herein is the generation of a large number of DC-tumor hybrid cells by electrofusion. Compared to other methods, electrofusion is reproducible and fusion rate is high. Therefore, additional purification or isolation may not be necessary for active immunization. Vaccination with fusion cells has provided a means to present both known and undefined tumor-associated Ags to both MHC class I and II-restricted pathways. A single dose of immunization with autologous fusion cells induced the rejection of established pulmonary metastases resulting in prolongation of life of the treated mice. The principle and methodology described here provide a strong impetus for clinical application of this approach for the treatment of cancer patients. Because of their ability to process and present natural tumor Ags to both CD4 and CD8 T cells, the use of DC-tumor fusion cells may also allow the identification of novel Ags, especially those reactive with CD4 T cells.

Heterokaryon fusion cells were plastic adherent, thus facilitating their separation from unfused DCs. The immunogenicity and therapeutic potential of fusion hybrids were analyzed in many murine model systems In vitro analyses revealed that while fusion hybrids stimulated specific IFN-γ secretion from both CD4 and CD8 immune T cells. Taken together, our results demonstrate the superior immunogenicity of DC-tumor fusion. The current technique of electrofusion is adequately developed for clinical use in cancer immunotherapy.

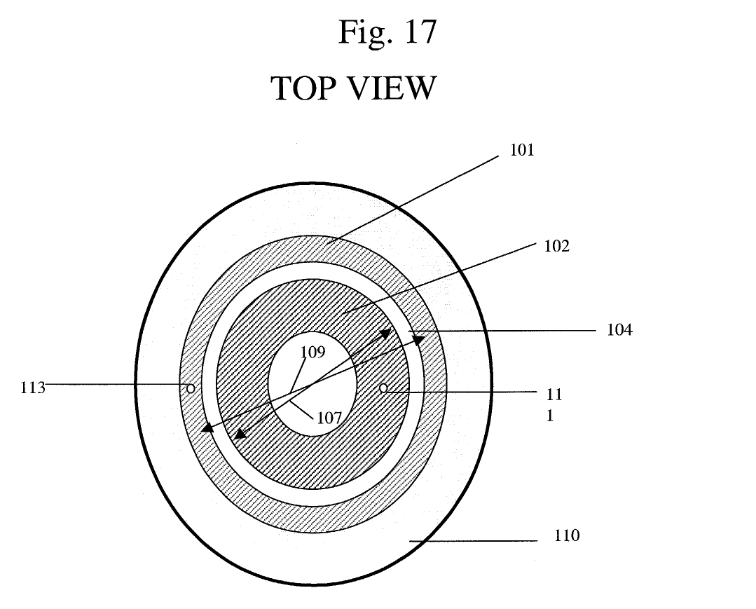

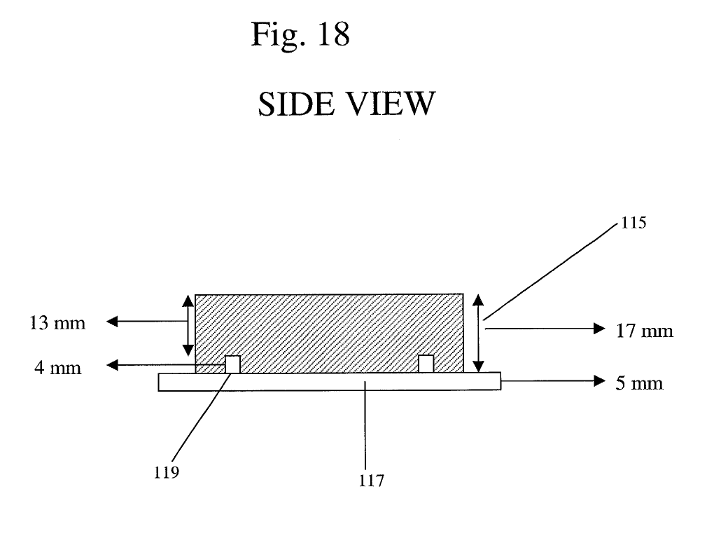

Finally FIGS. 17 and 18 illustrate an electrofusion chamber we designed in accordance with the present invention. This design has concentric chambers and currently is used. Our initial construction was two pieces of stainless steel put together on a glass microscope slide with silicon bathtop sealer. The two electrodes of this chamber had a gap of 5 mm. A variety of chamber designs with different gaps between two electrodes may be used. Preferably in this design the gap is 2-5 mm. Preferably they are designed with a circular configuration to promote inhomogeneity between the two electrodes and to process large numbers of cells (up to 300×10⁶). A variety of materials, including different stainless steels and 24 k gold electroplated ABS plastics, may be used. As shown in FIG. 17, an inner electrode 102 and an outer electrode 101 are spaced a predetermined distance apart forming the gap 104. The electrodes in this example are circular. The outer diameter 107 of the inner electrode 102 and an inner diameter 109 of the outer electrode form walls for the fusion chamber 101. The base 110 supports the two electrodes 101 and 102. Both electrodes have circuit connects 111, and 113 to connect the device to an electrical supply. In the example provided the gap 104 is about 3.5 mm. FIG. 18 illustrates that the height of the exemplary embodiment in FIG. 17. The floor 119 of the gap 104 is about 4 mm high in this example and is shown via cross-section.

It should be understood that while the invention has been described in detail herein, the examples were for illustrative purposes only. Other modifications of the embodiments of the present invention that are obvious to those of ordinary skill in the art of molecular and cellular biology, biophysics, and related disciplines are intended to be within the scope of the appended claims.

What is claimed:

1. A cancer vaccine comprised of:
a dendritic cell; and
a cancer cell, wherein said dendritic cell and said cancer cell are electrofused to thereby form a dendritic cell-cancer cell hybrid capable of stimulating an immune response.

2. The cancer vaccine of claim 1, wherein said dendritic cell is substantially MHC matched to a patient to be vaccinated.

3. The cancer vaccine of claim 1, wherein said dendritic cell is autologous for a patient to be vaccinated.

4. The cancer vaccine of claim 2, wherein said cancer cell shares Ag properties with the patient.

5. The cancer vaccine of claim 4, wherein, said cancer cell is allogenic to the patient.

6. The cancer vaccine of claim 4, wherein said cancer cell is autologous to the patient.

7. The cancer vaccine of claim 1, wherein the cancer cell is selected from the group consisting of a lung, brain, and skin cancer cell.

8. The cancer vaccine of claim 1, wherein said cancer cell and dendritic cell are human in origin.

9. A method for producing a plurality of dendritic cell-tumor cell hybrids useful for the induction of an anti-tumor response in a mammalian subject, said method comprising:
providing a sample of a tumor from a tumor source against which said response is needed; and
preparing a primary cell culture comprising tumor cells derived from said tumor sample or use of selected allogeneic tumors; and
preparing a primary cell culture comprising tumor cells derived from said tumor sample; and
providing HLA-compatible dendritic-like cells; and
electrofusing said dendritic-like cells with said tumor cells to produce a plurality of dendritic-like cell-tumor cell hybrids.

10. The method of claim 7, wherein said tumor source is selected from the group consisting of a purified surgical specimen, a short term cultured established tumor cell line, an allogeneic tumor cell that shares antigen properties with the subjects tumor, and an autologous tumor cell;

11. The method of claim 10, further including the step of isolating the DC-tumor cell hybrids based upon adherence to a plastic material.

12. The method of claim 10, wherein said dendritic-like cell is autologous to the subject.

13. A method for producing an anti-tumor response in a mammalian subject in need of anti-tumor treatment, said method comprising administering to said subject a plurality of electrofused DC-tumor cell hybrids.

14. A dendritic cell-tumor cell hybrid comprised of an electrofused dendritic cell-tumor cell capable of inducing an anti-tumor response.

15. The dendritic cell-tumor cell hybrid of claim 14, wherein said anti-tumor response occurs in vivo.

16. The dendritic cell-tumor cell hybrid of claim 14, wherein said anti-tumor response occurs in vitro.

17. The dendritic cell-tumor cell hybrid of claim 14, wherein said electrofused dendritic cell-tumor cell is useful in treating a tumor selected from the group consisting of a lung tumor, skin tumor, and brain tumor.

18. A method of treating, preventing, or ameliorating a tumor in a mammalian subject comprised of:
preparing a sample containing a plurality of cells of the tumor;
exposing the sample to dendritic cells of the patient;
electrofusing the cells of the tumor to the dendritic cells to thereby form dendritic cell-tumor cell hybrids; and
administering the dendritic cell-tumor cell hybrid to the patient to thereby prevent, treat, or ameliorate the tumor of the subject.

19. The method of claim 18, wherein said step of electrofusion occurs at a ratio of fused to unfused cells of greater than about 5%.

20. The method of claim 18, wherein said step of electrofusion occurs at a ratio of fused to unfused cells greater than 10%.

21. The method of claim 18, wherein said step of electrofusion occurs at a ratio of fused to unfused cells greater than 25%.

22. The method of claim 18, wherein said step of electrofusion occurs at a ratio of fused to unfused cells greater than 50%.

23. The method of claim 18, wherein said dendritic cell is substantially MHC matched to a patient to be vaccinated.

24. The method of claim 18, wherein said dendritic cell is autologous to a patient to be vaccinated.

25. The method of claim 18, wherein said cancer cell shares Ag properties but are allogeneic to the patient.

26. The method of claim 18, wherein the cancer cell is selected from the group consisting of a lung, brain, and skin cancer cell.

27. The method of claim 18, wherein said cancer cell and dendritic cell are human in origin.
